(19) United States
(12) Patent Application Publication
(10) Pub. No.: US 2006/0165639 A1
(43) Pub. Date: Jul. 27, 2006

(54) Use of cross-linked cationic polymers in cosmetics

(75) Inventors: Werner Gauweiler, Lustadt (DE); Ludger Wegmann, Ludwigshafen (DE); Marc Leduc, Speyer (DE); Lysander Chrisstoffels, Limburgerhof (DE); Michael Gotsche, Mannheim (DE); Claudia Wood, Weinheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 10/525,232

(22) PCT Filed: Aug. 29, 2003
(86) PCT No.: PCT/EP03/09596

§ 371 Date: Feb. 22, 2005

(30) Foreign Application Priority Data
Sep. 4, 2002 (DE) 102 41 296

(51) Int. Cl. A61K 8/81

(52) U.S. Cl. 424/70.16

(57) ABSTRACT

The invention relates to the use of a cationic crosslinked polymer which is preparable by free-radical polymerization in the presence of salts and of protective colloids in cosmetics.

The present invention relates to a process for the preparation of aqueous dispersions of crosslinked cationic water-soluble or water-swellable polymers based on monoethylenically unsaturated monomers which contain a quaternized or quaternizable nitrogen atom, by free-radical polymerization in an aqueous salt solution in the presence of a protective colloid, and to their use in hair cosmetic formulations.

Cationic polymers are used as conditioning agents in cosmetic formulations. Requirements for hair conditioning agents are, for example, a marked reduction in the required combing force in wet and also in dry hair, good detangling upon first combing and good compatibility with further formulation components. Furthermore, cationic polymers prevent electrostatic charging of the hair.

In shampoos, primarily cationic cellulose derivatives (polyquaternium-10) or guar gum derivatives are used. However, a build-up effect is observed with these compounds, i.e. the hair becomes coated with the conditioner upon repeated use and feels weighed down.

For the conditioning and setting of keratin substances, such as hair, nails and skin, synthetic polymers have also been used for years. In addition, synthetic polymers are used in cosmetic formulations which comprise pigments or cosmetically active components, as compatibility promoters for achieving a homogeneous, stable formulation.

For example, copolymers of acrylamide and dimethyldiallylammonium chloride (polyquaternium 7) are used. However, these have the disadvantage of high residual monomer contents since acrylamide and dimethyldiallylammonium chloride have unfavorable copolymerization parameters.

Despite extensive efforts, there continues to be a need for improvement with polymers for producing elastic hairstyles with simultaneously strong hold even under high atmospheric humidity, good ability to be washed out and good feel of the hair. The need for improvement likewise consists in the case of polymers for producing readily combable, detanglable hair and for conditioning skin and hair, in their sensorily perceivable properties, such as feel, volume, handleability, etc. In addition, clear aqueous preparations of these polymers are desired which are accordingly characterized by good compatibility with other formulation constituents.

Furthermore, there is a need for polymers which are suitable as conditioning agents for cosmetic preparations and which can be prepared with a high solids content. Of particular interest are polymers which have a high solids content, have a low viscosity whilst simultaneously retaining the application properties (such as, for example, combability).

It is an object of the present invention to find a cationic conditioning agent for cosmetic preparations, in particular shampoos, which does not have the disadvantages mentioned.

Further quaternized polymers and their use as conditioning agents in haircare formulations are known.

Thus, for example, EP-A-0 246 580 describes the use of uncrosslinked homopolymers and copolymers of 3-methyl-1-vinylimidazolium chlorides in cosmetic compositions. EP-A-0 544 158 and U.S. Pat. No. 4,859,756 claim the use of uncrosslinked homopolymers and copolymers of chloride-free, quaternized N-vinylimidazoles in cosmetic preparations. EP-A-0 715 843 discloses the use of uncrosslinked copolymers of a quaternized N-vinylimidazole, N-vinylcaprolactam and N-vinylpyrrolidone, and optionally a further comonomer in cosmetic preparations.

DE-A-31 06 974 describes a hair treatment composition of the preshampooing type which comprises uncrosslinked homopolymers and copolymers of quaternized diallylammonium compounds. DE-A-28 21 239 (U.S. Pat. No. 4,348,380) describes copolymers of quaternized diallylammonium compounds in hair cosmetic preparations. U.S. Pat. No. 5,275,809, EP-A-0 522 755, EP-A-0 521 665 and EP-A-0 521 666 disclose copolymers with dimethyldiallylammonium chloride for use in shampoos. In none of the abovementioned specifications is a crosslinked polymer described.

In addition, crosslinked cationic copolymers and their use as water-soluble and water-insoluble additives in very diverse areas are also described.

U.S. Pat. No. 4,806,345 describes crosslinked cationic thickeners for cosmetic formulations of quaternized dimethylaminoethyl methacrylate and acrylamide.

WO 93/25595 describes crosslinked cationic copolymers based on quaternized dialkylaminoalkyl acrylates or dialkylaminoalkylacrylamides. A proposed application is the use of these crosslinked copolymers as thickeners in cosmetic preparations.

DE-A-32 09 224 describes the preparation of crosslinked polymers based on N-vinylpyrrolidone and (quaternized) N-vinylimidazole. These polymers are claimed for use as adsorbents and ion exchangers.

Crosslinked, agglomerated vinylimidazole copolymers are specified in WO 96/26229 as dye transfer inhibitors. They are highly crosslinked, water-insoluble, barely swellable and therefore unsuitable for cosmetic formulations.

WO 96/37525 describes the preparation of crosslinked copolymers of, inter alia, N-vinylpyrrolidone and quaternized vinylimidazoles in the presence of polymerization regulators and their use, in particular in detergents.

U.S. Pat. No. 4,058,491 discloses crosslinked cationic hydrogels of N-vinylimidazole and N-vinylpyrrolidone and a quaternized basic acrylate, and further comonomers. These gels are proposed for the complexing and controlled release of anionic active substances.

DE-A-42 13 971 describes copolymers of an unsaturated carboxylic acid, quaternized vinylimidazole and optionally further monomers and a crosslinker. The polymers are proposed as thickeners and dispersants.

The use of copolymers with an aminoalkyl (meth)acrylate in cosmetics is described in EP-A-0 671 157. The polymers mentioned there are, however, used exclusively for the combined application with setting or conditioning polymers.

WO 97/35544 describes the use of crosslinked cationic polymers with dialkylaminoalkyl (meth)acrylates or dialkylaminoalkyl(meth)acrylamides in shampoo compositions.

EP-A-0 893 117 and EP 1064924 describes the use of high molecular weight crosslinked cationic polymers as solution polymers. These have a good conditioning effect in shampoos.

DE-A-197 31 907 describes the use of crosslinked cationic copolymers which contain N-vinylimidazoles in hair cosmetic formulations.

A disadvantage of these abovementioned inventions is that the preparation of these polymers takes place in the form of solutions with a very low solids content because otherwise the viscosities of these solutions are too high. An additional disadvantage is the generation of a relatively large proportion of undissolved gel particles. This leads to a large number of application disadvantages, such as, for example, longer polymerization times, prolonged filtration and bottling times. Due to the low solids content, high costs arise during the prepartion (reactor capacities) and high transportation costs. The gel particles produce application disadvantages. An undesired structure in the flow behavior of the shampoo formulation may be observed.

An alternative possibility is the use of a process for the preparation of water-soluble or water-swellable polymers in a W/O emulsion. This is claimed in EP-A-0 126 528 and comprises polymerizing the water-soluble monomers in the presence of emulsifiers with the addition of a special dispersion system consisting of alkanols. Use is also made inter alia of cationic comonomers. The oil phase used is aliphatic and aromatic hydrocarbons or higher aliphatic esters. The polymers are not intended for cosmetics applications.

The preparation of such polymers takes place according to the prior art by free-radical homopolymerization or copolymerization in either a homogeneous or heterogeneous phase. However, homogeneous solution polymerization leads, even in the case of low polymer concentrations, to high viscosities which, due to poor space-time yields, leads to high production costs. Polymerization in heterogeneous phase, such as, for example, polymerization in W/O emulsion in an organic solvent, may produce higher solids contents but with the disadvantage that an organic solvent is used which is undesired for cosmetic preparations for ecological and toxicological reasons. The disadvantages of a polymerization in heterogeneous phase using an organic solvent can be circumvented by preparing aqueous dispersions of water-soluble polymers.

An overview of aqueous dispersions of cationic polymers is given, for example, in WO 98/14405 and WO 98/31748.

In many embodiments, salts are used in order to precipitate out the polymers formed (WO 98/14490). The precipitated polymer is then stabilized with a suitable protective colloid. Without the corresponding protective colloid, the precipitated polymers have a tendency to stick together and to form a mass which is difficult to handle. Ideally, the end product should be an aqueous dispersion of a water-soluble or water-swellable cationic polymer which has a low viscosity despite a high solids content.

WO 99/46207 describes, for example, the preparation of an aqueous dispersion of a high molecular weight cationic polymer. Salts or combinations of salts are likewise used as cationic protective colloids.

Water-in-water emulsions of cationic acrylates and acrylamides in the presence of salts are described in EP 637 581. Here, use is made of cationic homopolymers or copolymers of cationic and neutral monomers as protective colloids.

WO 98/14490 describes cationic polymers or copolymers of cationic and neutral monomers or copolymers of neutral and anionic monomers for increasing the stability of emulsions.

WO 98/31748 describes the preparation of aqueous dispersions of uncrosslinked cationic water-soluble copolymers based on dimethylaminoethylmethacrylamide (DMAEMA) and diallydimethylammonium chloride (DADMAC) and neutral monomers such as acrylamide. For the stabilization, polyethylene glycol and polyamines are used.

WO 98/14405 describes suspensions of uncrosslinked water-soluble copolymers by polymerization of cationic methacrylamides and hydrophobic monomers using cationic polymers as protective colloid.

Further uncrosslinked aqueous dispersions of cationic polymers are described in DE 198 51 024 A1 and in WO 97/30094.

WO 99/46207 describes the preparation of aqueous dispersions of uncrosslinked high molecular weight amphoteric polymers which are carried out by copolymerization of anionic and cationic monomers in the presence of cationic or anionic protective colloids. In all of the examples mentioned, the polymerizations were carried out in the presence of salts.

U.S. Pat. No. 6,019,904 describes a process for the deinking of paper using a cationic polymer which is prepared in the presence of salt and a protective colloid (dispersant).

WO 02/34796 describes a preparation process for aqueous dispersions of polymers in the presence of protective colloids and one or more salts, the salt being added in two or more portions during the polymerization.

It is an object of the present invention to provide a polymerization process with improved space-time yields for preparing crosslinked cationic polymers with a high molecular weight. In addition, it is an object of the present invention to reduce the proportions of gel particles in order to thereby eliminate an undesired structure in the cosmetic formulation.

The aqueous dispersions preparable thereby have a high solids content and also readily handleable viscosities, are free from gel particles and are therefore highly suitable for hair cosmetic applications, for example as conditioner.

The cationic crosslinked polymers used according to the invention are obtainable by free-radically initiated polymerization of
a) 1 to 99.9% by weight, based on the total amount of monomers used for the preparation of the polymer, of at least one cationic or cationogenic vinyl group-containing monomer chosen from the group consisting of N-vinylimidazoles, diallylamines, dialkylaminoalkyl(meth)acrylamides and dialkylaminoalkyl(meth)acrylamides and dialkylaminoalkyl (meth)acrylates,
b) 0 to 99% by weight, based on the total amount of monomers used for the preparation of the polymer, of at least one neutral or basic water-soluble monomer different from (a),
c) 0 to 50% by weight, based on the total amount of monomers used for the preparation of the polymer, of at least one unsaturated acid or one unsaturated anhydride,
d) 0 to 50% by weight of at least one free-radically copolymerizable monomer different from (a), (b) or (c), and
e) 0.1 to 10% by weight, based on the total amount of monomers used for the preparation of the polymer, of at least one crosslinking monomer with at least two ethylenically unsaturated, nonconjugated double bonds,
   where the amounts a) to e) are chosen such that the resulting polymer, optionally after quaternization or protonation, has an overall positive charge,
   in water in the presence of
f) 1 to 100% by weight of the saturation amount in the reaction medium of one or more organic or inorganic salts, and
g) 0.1 to 30% by weight, based on the total weight of the dispersion, of at least one water-soluble protective colloid with a composition different from a) to e), and
   subsequent at least partial quaternization for cases where the monomer (a) is not quaternized.

Suitable monomers (a1) are chosen from one of the following groups:
N-vinylimidazole derivatives of the formula (I) 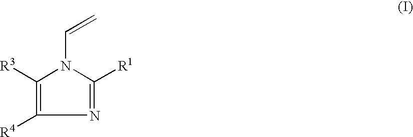
in which R¹, R² and R³, independently of one another, are hydrogen, C₁-C₄-alkyl or phenyl, preferably 2-methyl-N-vinylimidazole or N-vinylimidazole
N,N-diallylamine of the formula (II), 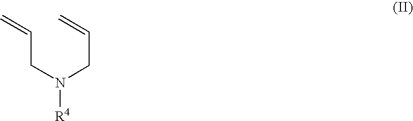
in which R⁴ is a C₁-C₂₄-alkyl radical, preferably N,N-diallyl-N-methylamine
N,N-diallylaminoalkyl derivatives of acrylic or methacrylic acid of the formula (III) 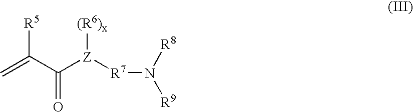
in which R⁵ and R⁶, independently of one another, are hydrogen or methyl, Z is a nitrogen atom where x=0, R⁷ is a linear or branched C₁-C₂₄-alkylene radical, and R⁸ and R⁹, independently of one another, are a C₁-C₂₄-alkylene radical.

Examples of compounds of the formula (I) are given in table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

Further monomers of the formula (I) which can be used are the ethyl, propyl or butyl analogs of the methyl-substituted 1-vinylimidazoles listed in table 1.

Examples of compounds of the formula (II) are diallylamines, in which R⁴ is methyl, ethyl, iso- or n-propyl, iso-, n- or tert-butyl, pentyl, hexyl, heptyl, octyl, nonyl or decyl. Examples of longer-chain radicals R⁴ are undecyl, dodecyl, tridecyl, pentadecyl, octadecyl and icosyl.

Suitable monomers of the formula (III) are, for example, N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]methacrylamide, N-[3-(diethylamino)propyl]methacrylamide or N-[3-(diethylamino)propyl]acrylamide, or mixtures thereof.

Preferred monomers (a) are 3-methyl-1-vinylimidazolium chloride and methosulfate, dimethyldiallylammonium chloride, and N,N-dimethylaminoethyl methacrylate and N-[3-(dimethylamino)propyl]methacrylamide, which have optionally been quaternized by methyl chloride, dimethyl sulfate or diethyl sulfate.

Particularly preferred monomers (a) are 3-methyl-1-vinylimidazolium chloride and methosulfate and dimethyldiallylammonium chloride and very particular preference is given to 3-methyl-1-vinylimidazolium chloride and methosulfate.

It is also possible to use mixtures of the monomers (a).

The monomers (a) are used for the application according to the invention in amounts of from 1 to 99.9% by weight, preferably from 5 to 70% by weight and very particularly preferably from 10 to 50% by weight. The % by weight amount refers to the total amount of monomers (a) to (e) used for the preparation of the polymer.

The monomers (a) can be used either in quaternized form as monomers or be polymerized in nonquaternized form, where, in the latter case, the resulting copolymer is either quaternized or protonated. In cases where the monomers are used in quaternized form, they can be used either as dried substance or in the form of concentrated solutions in solvents suitable for the monomers, for example in polar solvents, such as water, methanol, ethanol, acetone or electrolyte solutions.

Suitable for the protonation are, for example, mineral acids, such as HCl, H₂SO₄, and monocarboxylic acids, e.g. formic acid and acetic acid, dicarboxylic acids and polyfunctional carboxylic acids, e.g. oxalic acid and citric acid, and all other proton-releasing compounds and substances which are able to protonate the corresponding nitrogen atom. In particular, water-soluble acids are suitable for the protonation.

The protonation of the polymer can either be carried out after the polymerization or during the formulation of the cosmetic preparation, during which a physiologically compatible pH is usually set.

Protonation is understood as meaning that at least some of the protonatable groups of the polymer, preferably 20 to 100%, is protonated, resulting in an overall cationic charge of the polymer.

Suitable for the quaternization of the compounds of the formulae (I) to (III) are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic monomers of the formulae (I) to (III) can also be carried out with alkylene oxides, such as ethylene oxide or propylene oxide, in the presence of acids.

The quaternization of the monomer or of a polymer with one of said quaternization agents can take place by generally known methods.

Preferred quaternizing agents are: methyl chloride, dimethyl sulfate or diethyl sulfate.

As monomers of group (b), preference is given to those compounds which dissolve in water to an amount of more than 5% by weight at a temperature of 25° C. If they comprise polymeric monomers of group (b), then they may be present in amounts up to 98.98% by weight. Particularly preferably, they are present in amounts of from 22 to 97.98% by weight, in particular from 45 to 85% by weight.

Suitable water-soluble monomers (b) different from (a) are N-vinyllactams, e.g. N-vinylpiperidone, N-vinylpyrrolidone and N-vinylcaprolactam, N-vinylacetamide, N-methyl-N-vinylacetamide, N-vinylformamide, acrylamide, methacrylamide, N,N-dimethylacrylamide, N-methylolmethacrylamide, N-vinyloxazolidone, N-vinyltriazole, hydroxyalkyl (meth)acrylates, e.g. hydroxyethyl (meth)acrylate and hydroxypropyl (meth)acrylates, or alkyl ethylene glycol (meth)acrylates with 1 to 50 ethylene glycol units in the molecule.

Particular preference is given to using N-vinyllactams as monomers (b). Very particular preference is given to N-vinylpyrrolidone.

The monomers (b) are used for the application according to the invention in amounts of from 0.1 to 99% by weight, preferably 10 to 95% by weight and very particularly preferably 40 to 90% by weight. The % by weight amount refers to the total amount of monomers a) to e) used for the preparation of the polymer.

Suitable monomers (c) different from monomers (a) and (b) are C₁-C₄₀-alkyl esters of (meth)acrylic acid, where the esters are derived from linear, branched-chain or carbocyclic alcohols, e.g. methyl (meth)acrylate, ethyl (meth)acrylate, tert-butyl (meth)acrylate, isobutyl (meth)acrylate, n-butyl (meth)acrylate, stearyl (meth)acrylate, or esters of alkoxylated fatty alcohols, e.g. C₁-C₄₀-fatty alcohols, reacted with ethylene oxide, propylene oxide or butylene oxide, in particular C₁₀-C₁₈-fatty alcohols, reacted with 3 to 150 ethylene oxide units. Also suitable are N-alkyl-substituted acrylamides with linear, branched-chain or carbocyclic alkyl radicals, such as N-tert-butylacrylamide, N-butylacrylamide, N-octylacrylamide, N-tert-octylacrylamide.

Also suitable are styrene, vinyl esters and allyl esters of C₁-C₄₀-carboxylic acids, which may be linear, branched-chain or carbocyclic, e.g. vinyl acetate, vinyl propionate, vinyl neononanoate, vinyl neoundecanoic acid, vinyl t-butylbenzoate, alkyl vinyl ethers, for example methyl vinyl ether, ethyl vinyl ether, butyl vinyl ether, stearyl vinyl ether.

Acrylamides, such as N-tert-butylacrylamide, N-butylacrylamide, N-octylacrylamide, N-tert-octylacrylamide and N-alkyl-substituted acrylamides with linear, branched-chain or carbocyclic alkyl radicals, where the alkyl radical can have the meanings given above for R⁴.

Suitable monomers (c) are, in particular, C₁- to C₂₄-, very particularly C₁- to C₁₀, alkyl esters of (meth)acrylic acid, e.g. methyl (meth)acrylate, ethyl (meth)acrylate, tert-butyl (meth)acrylate, isobutyl (meth)acrylate, n-butyl (meth)acrylate and acrylamides, such as N-tert-butylacrylamide or N-tert-octylacrylamide.

If they comprise polymeric monomers of group (c), they may be present therein in amounts of up to 50% by weight, in particular up to 40% by weight, preferably up to 30% by weight.

Suitable crosslinkers (monomers (e)) are, for example, acrylic esters, methacrylic esters, allyl ethers or vinyl ethers of at least dihydric alcohols. The OH groups of the parent alcohols may be completely or partially etherified or esterified; however, the crosslinkers contain at least two ethylenically unsaturated groups.

Examples of the parent alcohols are dihydric alcohols, such as 1,2-ethanediol, 1,2-propanediol, 1,3-butanediol, 2,3-butanediol, 1,4-butanediol, but-2-ene-1,4-diol, 1,2-pentanediol, 1,5-pentanediol, 1,2-hexanediol, 1,5-hexanediol, 1,10-decanediol, 1,2-dodecanediol, 1,12-dodecanediol, neopentyl glycol, 3-methylpentane-1,5-diol, 2,5-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, mononeopentyl glycol hydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. As well as the homopolymers of ethylene oxide or propylene oxide, it is also possible to use block copolymers of ethylene oxide or propylene oxide or copolymers which contain ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. It is of course also possible to use the polyhydric alcohols following reaction with ethylene oxide or propylene oxide as the corresponding ethoxylates or propoxylates, respectively. The polyhydric alcohols can also be firstly converted into the corresponding glycidyl ethers by reaction with epichlorohydrin.

Further suitable crosslinkers are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polybasic carboxylic acids, for example malonic acid, tartaric acid, trimellitic acid, phthalic acid, terephthalic acid, citric acid or succinic acid.

As crosslinkers, it is likewise possible to use esters of unsaturated carboxylic acids with the polyhydric alcohols described above, for example maleic acid, crotonic acid, cinnamic acid or 10-undecenoic acid.

Also suitable are straight-chain or branched, linear or cyclic, aliphatic or aromatic hydrocarbons which have at least two double bonds, which in the case of aliphatic hydrocarbons must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable crosslinkers are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acids, methacrylic acid, itaconic acid, maleic acid, or at least dibasic carboxylic acids, as have been described above.

Further suitable as crosslinkers are triallylamine and triallylmonoalkylammonium salts, e.g. triallylmethylammonium chloride or methylsulfate.

Also suitable are N-vinyl compounds of urea derivatives, at least difunctional amides, cyanurates or urethanes, for example of urea, ethyleneurea, propyleneurea, or tartramide, e.g. N,N′-divinylethyleneurea or N,N′-divinylpropyleneurea.

Also suitable are alkylenebisacrylamides, such as methylenebisacrylamide and N,N′-(2,2-)butane and 1,1′-bis(3,3′-vinylbenzimidazolith-2-one)-1,4-butane.

Other suitable crosslinkers are, for example, alkylene glycol di(meth)acrylates, such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, pentaerythritol triallyl ether, and mixtures of the crosslinkers.

Further suitable crosslinkers are divinyldioxane, tetraallylsilane or tetravinylsilane.

Crosslinkers which are used particularly preferably are, for example, methylenebisacrylamide, triallylamine and triallylalkylammonium salts, pentaerythritol triallyl ether, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin. Very particularly preferred crosslinkers are methylenebisacrylamide, N,N′-divinylethyleneurea and acrylic esters of glycol, butanediol, trimethylolpropane or glycerol or acrylic esters of glycol, butanediol, trimethylolpropane or glycerol reacted with ethylene oxide and/or epichlorohydrin.

Very particularly preferred crosslinkers are pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

It is of course also possible to use mixtures of the abovementioned compounds. The crosslinker is preferably soluble in the reaction medium. If the solubility of the crosslinker in the reaction medium is low, it may be dissolved in a monomer or in a monomer mixture, or else be metered in in dissolved form in a solvent which is miscible with the reaction medium. Particular preference is given to those crosslinkers which are soluble in the monomer mixture.

The solution viscosity of the polymers according to the invention can be influenced to a wide degree by means of the content of crosslinkers.

The crosslinkers e) are used for the application according to the invention in amounts of from 0.05 to 10% by weight, preferably 0.07 to 5% by weight and very particularly preferably 0.1 to 2.5% by weight. The % by weight amount refers to the total amount of monomers a) to e) used for the preparation of the polymer.

The salt is used in order to deposit the polymer as it rises in a separate phase and thus to reduce the overall viscosity of the aqueous dispersion. The polymerization of the water-soluble monomers gives particles of water-soluble polymer if thorough mixing is adequately provided for.

The choice of the corresponding salts depends on the polymer to be generated and the protective colloid used. The choice of type and amount of salt should be such that the polymer to be prepared is insoluble in the salt solution.

The salts to be used which can be used for the precipitation of the polymer are described in detail in WO 98/14405 and WO 00/20470, which are hereby expressly incorporated by reference.

Particularly suitable salts are inorganic salts, preferably cosmotropic ones, such as chlorides, sulfates, phosphates or hydrogenphosphates of metal ions or ammonium ions. Typical representatives are sodium sulfate, potassium sulfate, ammonium sulfate, magnesium sulfate, aluminum sulfate, sodium chloride, calcium chloride, sodium dihydrogenphosphate, diammonium hydrogenphosphate, dipotassium hydrogenphosphate, calcium phosphate, sodium citrate and iron sulfate.

These salts can be used individually or as mixtures of two or more salts. Often a mixture of two or more salts is more effective than one salt on its own, based on the amount used.

Chaotropic salts, such as thiocyanates, perchlorates, chlorates, nitrates, bromides and iodides, can likewise be used. Typical representatives are calcium nitrate, sodium nitrate, ammonium nitrate, aluminum nitrate, sodium thiocyanate and sodium iodide.

The salts are added in an amount which is 1 to 100%, preferably 10 to 90% and particularly preferably 25 to 75%, of the saturation amount in the reaction medium.

100% saturation amount in the reaction medium is understood as meaning that amount of salt or salts which just still dissolve in the aqueous solution of the monomers used at the reaction temperature used without precipitation.

The polymeric water-soluble protective colloids in the water-in-water emulsions according to the invention are generally dissolved in the aqueous phase, although small amounts may be found in the dispersed phase. The amount of protective colloid in the continuous and dispersed phase can be determined using known analytical methods, such as Raman microscopy. In the absence of the protective colloid, no low-viscosity dispersion is formed, but a highly viscous gel is obtained.

The polymeric protective colloids contain at least one functional group chosen from ether, hydroxyl, carboxyl, sulfone, sulfate ester, ester, amino, amido, imino, tert-amino and/or quaternary ammonium groups. Examples of the protective colloids which may be mentioned are: cellulose derivatives, polyethylene glycol, polypropylene glycol, copolymers of ethylene glycol and propylene glycol, polyvinyl acetates, polyvinyl alcohol, starch and starch derivatives, guar gum and guar gum derivatives, dextran, polyvinyl 2-methylsuccinimide, polyvinyl 1,3-oxazolidone-2, polyvinyl-2-methylimidazoline, and copolymers which, as well as combinations of monomeric building blocks of the abovementioned polymers can contain, for example, the following monomer units: maleic acid, maleic anhydride, fumaric acid, itaconic acid, itaconic anhydride, (meth)acrylic acid, salts of (meth)acrylic acid or (meth)acrylamide compounds.

As neutral protective colloids, preference is given to using polyalkylene ethers, such as, for example, polyethylene glycol, polypropylene glycol or polybutylene 1,4-ether and starch and starch derivatives. The preparation of polyalkylene ethers is known, for example, from Kirk-Othmer, Encyclopedia of Chemical Technology, 3^{rd} Ed., Vol. 18, pp. 616-670, 1982, Wiley Interscience. Particularly preferred neutral protective colloids are polyalkylene ethers, such as, for example, polyethylene glycol, polypropylene glycol or polybutylene 1,4-ether.

Preferred protective colloids are homopolymers or copolymers of the following monomers:

a) Anionic Monomers:

acrylic acid, methacrylic acid, ethacrylic acid, maleic acid, itaconic acid, 2-acrylamido-2-methylpropanesulfonic acid, vinylsulfonic acid, vinylsulfuric acid, vinylphosphoric acid, styrenesulfonic acid, styrenesulfuric acid and ammonium and alkali metal salts thereof. Anionic radicals can also be formed after the reaction by hydrolysis of (meth)acrylamide or (meth)acrylate units.
   b) Cationic Monomers:
Preference is given to using vinylimidazoles, dialkylaminoalkyl (alk)acrylates, dialkylaminoalkylacrylamides, diallylalkylammonium and vinylamine quaternized with dimethyl sulfate, diethyl sulfate, or MeCl. The cationic charge can also be produced by after-treatment of the polymer, for example by quaternization (with methyl chloride or dimethyl sulfate, diethyl sulfate) or by protonation of the monomers or by hydrolysis, for example of vinylformamide to give vinylamine.
   c) Neutral Water-Soluble Monomers:
N-Vinylpyridines, N-vinylacetamide, N-vinylpyrrolidone, hydroxyalkyl (meth)acrylates, acrylamide, methacrylamide, VFA, PEG-acrylates and methacrylate derivatives, acrylates and acrylamides with an additional nitrogen function, such as DMAEMA, N-vinylcaprolactam.
   d) Neutral Hydrophobic Monomers:
Acrylates and styrenes with low solubility in water in as far as these sparingly water-soluble units do not render the resulting polymer overall water-insoluble or water-unswellable. Examples which may be mentioned are: butadiene, α-alkenes, vinylcyclohexane, vinyl halides, acrylonitrile, alkyl (alk′)acrylates or aryl (alk)acrylates in which the alkyl or aryl group has about 1 to 12 carbon atoms, such as methyl (meth)acrylate, ethyl (meth)acrylate, propyl (meth)acrylate, butyl (meth)acrylate, hexyl (meth)acrylate, ethylhexyl (meth)acrylate, isoalkyl (meth)acrylate, cyclohexyl (meth)acrylate or aromatic (meth)acrylates or alkyl or aryl(alk)acrylamides in which the alkyl or aryl group carries about 1 to 12 carbon atoms, such as methylmeth(acrylamide), ethylmeth(acrylamide), ethylmeth(acrylamide), t-butylmeth(acrylamide), dimethylmeth(acrylamide), hexylmeth(acrylamide), ethylhexylmeth(acrylamide), isoalkylmeth(acrylamide), cyclohexylmeth(acrylamide), or aromatic meth(acrylamides), t-butylacrylamide.

As protective colloids, particular preference is given to using neutral water-soluble polymers, for example polyvinyl alcohol, polyvinylformamide, polyvinylpyrrolidone, poly(meth)acrylamide, polyhydroxyalkyl (meth)acrylate, and homopolymers and copolymers of the monomers under c) and d).

As protective colloids, particular preference is given to using polyelectrolytes, such as, for example, polymers, comprising monomer building blocks, such as, for example, salts of (meth)acrylic acid as anionic monomer building blocks or methyl chloride-quaternized derivatives of N,N-dimethylaminoethyl (meth)acrylate, N,N-dimethylaminopropyl (meth)acrylate or N,N-dimethylaminohydroxypropyl (meth)acrylate or N,N-dimethylaminopropyl(meth)acrylamide, and homopolymers and copolymers of the monomers under a) and b), copolymers of the monomers a) and c), a) and d), b) and c), b) and d).

The overall charge of the polyelectrolyte can either be positive or negative. The polyelectrolyte should be water-soluble even if it is built up from monomers which are not particularly readily water-soluble.

The K values of the protective colloids are in a range between 10 and 350, preferably 20 to 200 and particularly preferably 35 to 110. The K values are measured in accordance with Fikentscher, Cellulosechemie, Vol. 13, pp. 58-64 (1932) at 25° C. as 0.1% strength in 5% by weight sodium chloride solution.

These protective colloids can be used individually or as mixtures of two or more protective colloids.

The protective colloids are used in amounts of from 0.1 to 30% by weight, preferably 0.5 to 20% by weight and particularly preferably 1 to 10% by weight, based on the total weight of the dispersion.

Total weight of the dispersion is understood as meaning the weight of the monomers used, of the water used and of the salts used.

Initiators which can be used for the free-radical polymerization are the water-soluble and water-insoluble peroxo and/or azo compounds, for example alkali metal or ammonium peroxydisulfates, hydrogen peroxide, dibenzoyl peroxide, tert-butyl perpivalate, 2,2′-azobis(2,4-dimethylvaleronitrile), tert-butyl peroxineodecanoate, tert-butyl per-2-ethylhexanoate, di-tert-butyl peroxide, tert-butyl hydroperoxide, azobisisobutyronitrile, azobis(2-amidinopropane)dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate. The initiators can be used in the customary amounts, for example 0.05 to 7% by weight, based on the amount of the monomers to be polymerized.

The co-use of redox coinitiators, for example benzoin, dimethylaniline and organically soluble complexes and salts of heavy metals, such as copper, cobalt, manganese, nickel and chromium or, in particular, iron, can reduce the half-life times of said peroxides, particularly of the hydroperoxides, meaning that, for example, tert-butyl hydroperoxide is effective in the presence of 5 ppm of copperII acetylacetonate even at 100° C.

Preference is given to using readily water-soluble initiators.

The polymerization reaction is started with the help of polymerization initiators which decompose into free radicals. It is possible to use all initiators which are known for the polymerization of the monomers. For example, initiators which decompose into free radicals and which have half-life times of less than three hours at the temperature chosen in the particular case are suitable. If the polymerization is carried out at different temperatures by carrying out initial polymerization of the monomers firstly at a lower temperature and then completing the polymerization at a significantly higher temperature, then use is expediently made of at least two different initiators which have an adequate rate of decomposition in the temperature range chosen in the particular case.

The polymerization is usually carried out at temperatures between 20 and 100° C., preferably between 30 and 90° C., 40 and 80° C., and at atmospheric pressure or under autogenous pressure.

The polymerization can optionally also be carried out in the presence of polymerization regulators in order to regulate the molecular weight of the polymers. If the aim is to prepare particularly low molecular weight copolymers, larger amounts of polymerization regulators are used, whereas for the preparation of high molecular weight copolymers only small amounts of polymerization regulators are used or the polymerization is carried out in the absence of these substances. Suitable polymerization regulators are, for example, 2-mercaptoethanols, mercaptopropanols, mercaptobutanols, thioglycolic acid, N-dodecylmercaptan, tert-dodecylmercaptan, thiophenol, mercaptopropionic acid, allyl alcohol and acetaldehyde. The polymerization regulators are used, based on the monomers used, in an amount of from 0 to 10% by weight, preferably 0 to 5% by weight, particularly preferably 0 to 2% by weight.

Customary processing auxiliaries, such as complexing agents, buffers, odorants, can be added where necessary. Viscosity modifiers, such as glycerol, methanol, ethanol, t-butanol, glycol, etc., can likewise be added to the aqueous dispersion.

The polymerization is carried out in a preferred embodiment as a batch procedure. In this connection, it is preferred to initially introduce the monomers (a-c) into the vessel.

In a preferred embodiment, the processes according to the invention are carried out as feed processes. Here, individual reactants or all of the reactants are added, completely or partially, in portions or continuously, together or in separate feeds, to a reaction mixture. It is, however, also possible to meter in the initiator to the initial charge heated to the polymerization temperature and comprising the salts, protective colloids, and monomers (a) to (e). In a further variant the initiator and the crosslinker (d) are added to a mixture of monomers (a) and optionally monomers (b) and (c) after the polymerization temperature has been reached. It is also possible to heat the initial charge comprising the salt and protective colloids to the polymerization temperature and to add initiator and monomers (d) in separate feeds or together. It is of course also possible to add initiator, monomers (d) and monomers (a) and optionally monomers (b) and (c) to an initial charge heated to the polymerization temperature and comprising a mixture of salts and protective colloids. Preference is given to using a mixture of salt and protective colloid in water or a mixture of salt or protective colloid in water and at least part of the monomers (a) and optionally (b) and (c), and optionally further components as the initial charge.

The dispersions are usually milky white and have a viscosity of from 100 to 50 000 mPas, preferably from 200 to 20 000 mPas, particularly preferably from 300 to 15 000 mPas.

The dispersions produced in the polymerization can, after the polymerization process, be subjected to a physical or chemical after-treatment. Such processes are, for example, the known processes for reducing residual monomers, such as, for example, after-treatment by adding polymerization initiators or mixtures of two or more polymerization initiators at suitable temperatures or heating the polymerization solution to temperatures above the polymerization temperature, an after-treatment of the polymer solution by means of steam or stripping with nitrogen or treatment of the reaction mixture with oxidizing or reducing reagents, adsorption processes, such as the adsorption of contamination on selected media, such as, for example, activated carbon, or an ultrafiltration. It can also be followed by the known work-up steps, for example suitable drying processes, such as spray-drying, freeze-drying or roll-drying, or agglomeration processes following the drying. The dispersions with a low content of residual monomers obtained by the process according to the invention can also be sold directly.

The polymers according to the invention can advantageously be used in cosmetic preparations, in particular hair cosmetic preparations.

The term “cosmetic preparations” is to be understood in the broad sense and means all those preparations which are suitable for application to skin and/or hair and/or nails and pursue a purpose other than a purely medicinal-therapeutic purpose.

The polymers according to the invention can be used in skin cosmetic preparations.

For example, the polymers according to the invention are used in cosmetic compositions for cleansing the skin. Such cosmetic cleansing compositions are chosen from bar soaps, such as toilet soaps, curd soaps, transparent soaps, luxury soaps, deodorizing soaps, cream soaps, baby soaps, skin protection soaps, abrasive soaps and syndets, liquid soaps, such as pasty soaps, soft soaps and washing paste, and liquid washing, shower and bath preparations, such as washing lotions, shower baths and shower gels, foam baths, oil baths and scrub preparations.

Preferably, the polymers according to the invention are used in cosmetic compositions for the care and protection of the skin, in nailcare compositions and in preparations for decorative cosmetics.

Particular preference is given to the use in skincare compositions, intimate care compositions, footcare compositions, deodorants, light protection compositions, repellants, shaving compositions, hair-removal compositions, anti-acne compositions, make-up, mascara, lipsticks, eyeshadows, kohl pencils, eyeliners, blushers, powders and eyebrow pencils.

The skincare compositions are in particular in the form of W/O or O/W skin creams, day and night creams, eye creams, face creams, antiwrinkle creams, moisturizing creams, bleaching creams, vitamin creams, skin lotions, care lotions and moisturizing lotions.

The polymers according to the invention can develop particular effects in the cosmetic preparations. The polymers can, inter alia, contribute to the moisturization and conditioning of the skin and to the improvement in the feel of the skin. The polymers can also act as thickeners in the formulations. The addition of the polymers according to the invention can, in certain formulations, bring about a considerable improvement in the skin compatibility.

The copolymers according to the invention are present in the skin cosmetic preparations in an amount of from about 0.001 to 20% by weight, preferably 0.01 to 10% by weight, very particularly preferably 0.1 to 5% by weight, based on the total weight of the composition.

Depending on the field of use, the compositions according to the invention can be applied in a form suitable for skincare, such as, for example, in the form of a cream, foam, gel, stick, powder, mousse, milk or lotion.

As well as the polymers according to the invention and suitable solvents, the skin cosmetic preparations can also comprise additives customary in cosmetics, such as emulsifiers, preservatives, perfume oils, cosmetic active ingredients, such as phytantriol, vitamin A, E and C, retinol, bisabolol, panthenol, light protection agents, bleaching agents, colorants, tinting agents, tanning agents (e.g. dihydroxyacetone), collagen, protein hydrolysates, stabilizers, pH regulators, dyes, salts, thickeners, gel formers, bodying agents, silicones, humectants, refatting agents and further customary additives.

Suitable solvents which may be mentioned are, in particular, water and lower monoalcohols or polyols having 1 to 6 carbon atoms or mixtures thereof; preferred monoalcohols or polyols are ethanol, isopropanol, propylene glycol, glycerol and sorbitol.

As further customary additives, fatty bodies may be present, such as mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin. It is of course also possible to use mixtures thereof.

Customary thickeners in such formulations are crosslinked polyacrylic acids and derivatives thereof, polysaccharides, such as xanthan gum, agar agar, alginates or tyloses, carboxymethylcellulose or hydroxycarboxymethylcellulose, fatty alcohols, monoglycerides and fatty acids, polyvinyl alcohol and polyvinylpyrrolidone.

The polymers according to the invention can also be mixed with customary polymers if specific properties are to be set.

Examples of suitable conventional polymers are anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinyl esters (e.g. Luviset® grades), maleic anhydride copolymers, optionally reacted with alcohols, anionic polysiloxanes, e.g. carboxyfunctional, copolymers of vinylpyrrolidone, t-butyl acrylate, methacrylic acid (e.g Luviskol® VBM), copolymers of acrylic acid and methacrylic acid with hydrophobic monomers, such as, for example, C₄-C₃₀-alkyl esters of meth(acrylic acid), C₄-C₃₀-alkylvinyl esters, C₄-C₃₀-alkylvinyl ethers and hyaluronic acid.

Further suitable polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7) and chitosan.

Suitable further polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethylenimines and salts thereof, polyvinylamines and salts thereof, cellulose derivatives, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The copolymers according to the invention are used in cosmetic preparations which are produced in accordance with the customary guidelines known to the person skilled in the art.

Such formulations are advantageously in the form of emulsions, preferably in the form of water-in-oil (W/O) or oil-in-water (O/W) emulsions. It is, however, also possible and in some cases advantageous according to the invention to choose other types of formulation, for example hydrodispersions, gels, oils, oleogels, multiple emulsions, for example in the form of W/O/W or O/W/O emulsions, anhydrous ointments or ointment bases etc.

The emulsions which can be used according to the invention are prepared by known methods.

As well as the copolymer according to the invention, the emulsions comprise customary constituents, such as fatty alcohols, fatty acid esters and, in particular, fatty acid triglycerides, fatty acids, lanolin and derivatives thereof, natural or synthetic oils or waxes and emulsifiers in the presence of water.

The selection of additives specific to the type of emulsion and the preparation of suitable emulsions is described, for example, in Schrader, Grundlagen und Rezepturen der Kosmetika [Fundamentals and Formulations of Cosmetics], Hüthig Buch Verlag, Heidelberg, 2nd edition, 1989, third part, to which express reference is hereby made.

Thus, a skin cream which can be used according to the invention can be in the form, for example, of a W/O emulsion. An emulsion of this type comprises an aqueous phase which is emulsified in an oil or fatty phase by means of a suitable emulsifier system.

The concentration of the emulsifier system in this type of emulsion is about 4 to 35% by weight, based on the total weight of the emulsion; the fatty phase constitutes about 20 to 60% by weight and the aqueous phase about 20 to 70% by weight, in each case based on the total weight of the emulsion. The emulsifiers are those which are customarily used in this type of emulsion. They are chosen, for example, from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start point under atmospheric pressure is at about 250° C. and whose distillation end point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

In order to favor the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by adding the fatty phase and the emulsifier to the batch container. The latter is heated at a temperature of from 70 to 75° C., then the oil-soluble ingredients are added, and water which has been heated beforehand to the same temperature and in which the water-soluble ingredients have been dissolved beforehand is added with stirring; the mixture is stirred until an emulsion of the desired fineness is achieved, which is then left to cool to room temperature, if necessary with a lesser amount of stirring.

In addition, a care emulsion according to the invention may be in the form of a O/W emulsion. Such an emulsion usually comprises an oil phase, emulsifiers which stabilize the oil phase in the water phase, and an aqueous phase which is usually present in thickened form.

The aqueous phase of the O/W emulsion of the preparations according to the invention optionally comprises
alcohols, diols or polyols and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are, preferably O/W emulsifiers, such as polyglycerol esters, sorbitan esters or partially esterified glycerides.

The preparation may be carried out by melting the oil phase at about 80° C.; the water-soluble constituents are dissolved in hot water, added to the oil phase slowly and with stirring; homogenized and stirred until cold.

The polymers according to the invention are also suitable for use in washing and shower gel formulations and bath preparations.

As well as the polymers according to the invention, such formulations usually comprise anionic surfactants as base surfactants and amphoteric and nonionic surfactants as cosurfactants, and also lipids, perfume oils, dyes, organic acids, preservatives and antioxidants, and thickeners/gel formers, skin conditioning agents and humectants.

In the washing, showering and bathing preparations, all anionic, neutral, amphoteric or cationic surfactants customarily used in body-cleansing compositions may be used.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable compounds are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, it is possible to use cocodimethylsulfopropylbetaine, laurylbetaine, cocamidopropylbetaine or sodium cocamphopropionate.

Suitable nonionic surfactants are, for example, the reaction products of aliphatic alcohols or alkylphenols, having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

In addition, the washing, shower and bathing preparations can comprise customary cationic surfactants, such as, for example, quaternary ammonium compounds, for example cetyltrimethylammonium chloride.

Additionally, further customary cationic polymers may also be used, such as, for example, copolymers of acrylamide and dimethyldiallylammonium chloride (Polyquaternium-7), cationic cellulose derivatives (Polyquaternium-4, -10), guar hydroxypropyltrimethylammonium chloride (INCI: Hydroxypropyl Guar Hydroxypropyltrimonium Chloride), copolymers of N-vinylpyrrolidone and quaternized N-vinylimidazole (Polyquaternium-16, -44, -46), copolymers of N-vinylpyrrolidone/dimethylaminoethyl methacrylate, quaternized with diethyl sulfate (Polyquaternium-11), and others.

In addition, the washing and shower gel formulations and bathing preparations can comprise thickeners, such as, for example, sodium chloride, PEG-55, propylene glycol oleate, PEG-120 methyl glucose dioleate and others, and also preservatives, further active ingredients and auxiliaries and water.

Hair cosmetic preparations include, in particular styling compositions and/or conditioning agents in hair cosmetic preparations, such as hair treatments, hair mousses, hair gels or hair sprays, hair lotions, hair rinses, hair shampoos, hair emulsions, hair-end fluids, neutralizers for permanent waves, hair colorants and bleaches, hot-oil treatment preparations, conditioners, setting lotions or hair sprays. Depending on the field of application, the hair cosmetic preparations can be applied in the form of (aerosol) spray, (aerosol) mousse, gel, gel spray, cream, lotion or wax.

In a preferred embodiment, the hair cosmetic formulations according to the invention comprise
a) 0.05 to 20% by weight of the polymer according to the invention
b) 20 to 99.95% by weight of water and/or alcohol
c) 0 to 79.5% by weight of further constituents

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolysates, alpha- and beta-hydroxycarboxylic acids, stabilizers, pH regulators, dyes, viscosity regulators, gel formers, salts, humectants, refatting agents, complexing agents and further customary additives.

Also included here are all styling and conditioning polymers known in cosmetics which can be used in combination with the polymers according to the invention if very specific properties are to be set.

Examples of suitable conventional hair cosmetic polymers are anionic polymers. Such anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset® P.U.R.) and polyureas. Particularly suitable polymers are copolymers of t-butyl acrylate, ethyl acrylate, methacrylic acid (e.g. Luvimer® 100P), copolymers of N-tert-butylacrylamide, ethyl acrylate, acrylic acid (Ultrahold® 8, strong), copolymers of vinyl acetate, crotonic acid and optionally further vinyl esters (e.g. Luviset® grades), maleic anhydride copolymers, optionally reacted with alcohols, anionic polysiloxanes, e.g. carboxyfunctional ones, copolymers of vinylpyrrolidone, t-butyl acrylate, methacrylic acid (e.g. Luviskol® VBM).

In addition, the group of polymers suitable for the combination with the polymers according to the invention includes, by way of example, Balance® CR (National Starch; acrylate copolymer), Balance® 0/55 (National Starch; acrylate copolymer), Balance® 47 (National Starch; octylacrylamide/acrylate/butylaminoethyl methacrylate copolymer), Aquaflex® FX 64 (ISP; isobutylene/ethylmaleimide/hydroxyethylmaleimide copolymer), Aquaflex® SF-40 (ISP/National Starch; VP/vinyl caprolactam/DMAPA acrylate copolymer), Allianz® LT-120 (ISP/Rohm & Haas; acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid) in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Acudyne® 258 (Rohm & Haas; acrylate/hydroxy ester acrylate copolymer), Luviset® PUR (BASF, polyurethane-1), Luviflex® Silk (BASF), Eastman® AQ48 (Eastman).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCI name polyquaternium, e.g. copolymers of vinylpyrrolidone/N-vinylimidazolium salts (Luviquat® FC, Luviquat® HM, Luviquat® MS, Luviquat® Care, copolymers of N-vinylpyrrolidone/dimethylaminoethyl methacrylate, quaternized with diethyl sulfate (Luviquat® PQ 11), copolymers of N-vinylcaprolactam N-vinylpyrrolidone/N-vinylimidazolium salts (Luviquat® Hold); cationic cellulose derivatives (Polyquaternium-4 and -10), acrylamide copolymers (Polyquaternium-7).

Cationic guar derivatives such as guar hydroxypropyltrimonium chloride (INCI) can also be used.

Further suitable hair cosmetic polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To set certain properties, the preparations may additionally also comprise conditioning substances based on silicone compounds. Suitable silicone compounds are, for example, polyalkylsiloxanes, polyarylsiloxanes, polyarylalkylsiloxanes, polyether siloxanes, silicone resins, or dimethicone copolyols (CTFA) and aminofunctional silicone compounds such as amodimethicones (CTFA).

The polymers according to the invention are particularly suitable as setting agents in hair styling preparations, in particular hair sprays (aerosol sprays and pump sprays without propellant gas) and hair mousses (aerosol mousses and pump mousses without propellant gas).

In a preferred embodiment, these preparations comprise
a) 0.1 to 10% by weight of the polymer according to the invention
b) 20 to 99.9% by weight of water and/or alcohol
c) 0 to 70% by weight of a propellant
d) 0 to 20% by weight of further constituents

Propellants are the propellants customarily used for hair sprays or aerosol mousses. Preference is given to mixtures of propane/butane, pentane, dimethyl ether, 1,1-difluoroethane (HFC-152 a), carbon dioxide, nitrogen or compressed air.

A formulation for aerosol hair mousses which is preferred according to the invention comprises
a) 0.1 to 10% by weight of the polymer according to the invention
b) 55 to 99.8% by weight of water and/or alcohol
c) 5 to 20% by weight of a propellant
d) 0.1 to 5% by weight of an emulsifier
e) 0 to 10% by weight of further constituents

The emulsifiers used may be emulsifiers customarily used in hair mousses. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, Quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation which is suitable according to the invention for styling gels may, for example, have the following composition:
a) 0.1 to 10% by weight of the polymer according to the invention
b) 60 to 99.85% by weight of water and/or alcohol
c) 0.05 to 10% by weight of a gel former
d) 0 to 20% by weight of further constituents

Gel formers which may be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, polyquaternium-32 (and) paraffinum liquidum (INCI), sodium acrylates copolymer (and) paraffinum liquidum (and) PPG-1 trideceth-6, acrylamidopropyl trimonium chloride/acrylamide copolymer, steareth-10 allyl ether acrylates copolymer, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

The polymers according to the invention can also be used in shampoo formulations as setting agents and/or conditioning agents. Suitable conditioning agents are, in particular, polymers with a cationic charge. Preferred shampoo formulations comprise
a) 0.05 to 10% by weight of the polymer according to the invention
b) 25 to 94.95% by weight of water
c) 5 to 50% by weight of surfactant
c) 0 to 5% by weight of a further conditioning agent
d) 0-10% by weight of further cosmetic constituents

In the shampoo formulations, it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable examples are sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, it is possible to use cocodimethylsulfopropylbetaine, laurylbetaine, cocamidopropylbetaine or sodium cocamphopropionate.

Examples of suitable nonionic surfactants are the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, alkyl polyglycosides or sorbitan ether esters.

Furthermore, the shampoo formulations may comprise customary cationic surfactants, such as, for example, quaternary ammonium compounds, for example cetyltrimethylammonium chloride.

In the shampoo formulations, to achieve certain effects, customary conditioning agents may be used in combination with the polymers according to the invention. These include, for example, cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4, and -10), acrylamide copolymers (Polyquaternium-7). It is also possible to use protein hydrolysates, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and aminofunctional silicone compounds such as amodimethicones (CTFA). It is also possible to use cationic guar derivatives, such as guar hydroxypropyltrimonium chloride (INCI).

The examples below serve to further illustrate the invention.

A Preparation of the Polymer

### EXAMPLE 1

### Water-in-Water Emulsion Polymerization of VP and Quat-311 in the Presence of a Mixture of Na₂SO₄ and NaCl

Sodium sulfate (50 g) and sodium chloride (30 g), vinylpyrrolidone (128 g), quat-311 (64 g, 50% by weight solution in water) and 0.35 g of butanediol diacrylate were dissolved in water (347 g). As protective colloid, 63 g of poly(vinylamine-co-acrylic acid=3:7) (25% by weight solution in water) were added. The pH was adjusted to a value of 6.75 using 50% sulfuric acid, and the emulsion obtained was flushed with nitrogen for about 10 minutes. The free radical initiator (2,2′-azobis(2-amidinopropane)dihydrochloride: V-50; 0.48 g) was then added and the reaction mixture was heated to 60° C. under a nitrogen atmosphere. After the reaction mixture had been stirred at this temperature for 3 hours, a second portion of V-50 (0.96 g) was added and the mixture was stirred for a further 3 hours at 70° C. and then cooled to room temperature. This gave a white suspension with a polymer content of 23% and a viscosity of 1650 mPas. Dilution of the emulsion with water to 6.5% by weight gave a clear solution of 8000 mPas.

### EXAMPLE 2

### Water-in-Water Emulsion Polymerization of VP and QVI in the Presence of Na₂SO₄

Sodium sulfate (77 g), vinylpyrrolidone (128 g), quaternised vinylimidazolium (64 g, 50% by weight solution in water) and 0.48 g of triallylamine were dissolved in water (347 g). As protective colloid, 63 g of poly(vinylamine-co-acrylic acid)=1:9) (25% by weight solution in water) were added. The pH was adjusted to a value of 6.75 using 50% sulfuric acid, and the emulsion obtained was flushed with nitrogen for about 10 minutes. The free radical initiator (V-50; 0.48 g) was then added and the reaction mixture was heated to 60° C. under a nitrogen atmosphere. After the reaction mixture had been stirred at this temperature for 3 hours, a second portion of V-50 (0.96 g) was added, and the mixture was stirred for a further 3 hours at 70° C. and then cooled to room temperature. This gave a white suspension with a polymer content of 23% and a viscosity of 1100 mPa·s. Dilution of the emulsion with water to 6.5% by weight gave a clear solution of 11 500 mPas.

### EXAMPLE 3

### Water-in-Water Emulsion Polymerization of VP and QVI in the Presence of a Mixture of Na₂SO₄ and (NH₄)₂SO₄

Sodium sulfate (65 g) and ammonium sulfate (30 g), vinylpyrrolidone (128 g), quaternized vinylimidazolium (64 g, 45% by weight solution in water) and 0.25 g of triallylamine were dissolved in water (347 g). As protective colloid, 63 g of polyacrylic acid (Sokalan HP80 25% by weight solution in water) were added. The pH was adjusted to a value of 6.75 using 50% sulfuric acid and the emulsion obtained was flushed with nitrogen for about 10 minutes. The free radical initiator (V-44; 0.48 g) was then added and the reaction mixture was heated to 60° C. under a nitrogen atmosphere. After the reaction mixture had been stirred at this temperature for 3 hours, a second portion of V-50 (0.96 g) was added and the mixture was stirred for a further 3 hours at 70° C. and then cooled to room temperature. This gave a white suspension with a polymer content of 23% and a viscosity of <1000 mPas. Dilution of the emulsion with water to 6.5% by weight gave a clear solution of 4500 mPas.

### EXAMPLE 4 (COMPARATIVE EXPERIMENT TO EXAMPLE 2)

### Solution Polymerization of VP and QVI

Vinylpyrrolidone (128 g), quaternized vinylimidazolium (64 g, 50% by weight solution in water) and 0.75 g of triallylamine were dissolved in water (347 g). The pH was adjusted to a value of 6.75 using 50% sulfuric acid and the emulsion obtained was flushed with nitrogen for about 10 minutes. The free radical initiator (V-50; 0.48 g in 200 g of water) was then added and the reaction mixture was heated to 65° C. under a nitrogen atmosphere. After the reaction mixture had been stirred at this temperature for 3 hours, a second portion of V-50 (0.96 g in 250 g of water) was added and the mixture was stirred for a further 3 hours at 70° C. and then cooled to room temperature. After the polymerization, 800 g of water were added in order to adjust the concentration to 6.5% by weight. This gave a clear solution with a polymer content of 6.5% and a viscosity of 6050 mPas.

### EXAMPLE 5 (COMPARATIVE EXPERIMENT TO EXAMPLE 3)

### Solution Polymerization of VP and QVI

Sodium sulfate (77 g), vinylpyrrolidone (128 g), quaternized vinylimidazolium (64 g, 45% by weight solution in water) and 0.25 g of triallylamine were dissolved in water (500 g). The pH was adjusted to a value of 6.75 using 50% sulfuric acid and the emulsion obtained was flushed with nitrogen for about 10 minutes. The free radical initiator (V-50; 0.48 g in 100 g of water) was then added and the reaction mixture was heated to 65° C. under a nitrogen atmosphere. After the reaction mixture had been stirred at this temperature for 3 hours, a second portion of V-50 (0.96 g in 100 g of water) was added and the mixture was stirred for a further 3 hours at 70° C. and then cooled to room temperature. This gave a clear solution with a polymer content of 22.8% and a viscosity of >16 500 mPas. The polymer comprised large amounts of insoluble solid particles and dilution of the solution with water to 6.5% by weight gave a solution of 1100 mPas with solid insoluble particles.

TABLE 1
Summary of experiments 1 to 6

| | Tel Quel. viscosity | Solution viscosity | Appearance |
|---|---|---|---|
| Example | (mPas/% by wt.) | (mPas/6.5% by wt.) | of solution |
| 1 | 1650/23/emulsion | 8000/solution | clear/no structure |
| 2 | 1100/23/emulsion | 11500/solution | clear/no structure |
| 3 | <500/23/emulsion | 4500/solution | clear/no structure |
| 4 | 950/6.5/solution | 950/solution | clear/slight struc- |
| | | | ture |
| 5 | 16400/23/solution | 1100/solution | clear/structure |

The polymers to be used according to the invention have, as aqueous solutions at 6.5% by weight, higher solution viscosities (compare example 1 to 3 and example 4 to 5).

The polymers can be prepared as dispersions with a relatively high solids content (23% by weight) and a low viscosity. The 23% by weight solution of polymer is not readily pumpable and has many gel particles, but in the form of a dispersion it can be handled easily (compare example 3 and example 5).

The polymeric solutions at 6.5% by weight exhibit, as solution polymer, a flow structure which is produced by the gel particles, but the solutions of the dispersions according to the invention exhibit no structure.

B Use of the Dispersions According to the Invention as Conditioners

For the performance investigation, the polymers were used in a surfactant solution formulation with the following composition:
40.0% of Texapon NSO (sodium laureth sulfate solution 28%; Cognis)
10.0% of Tego-Betaine L7 (cocamidopropylbetaine solution 30%; Goldschmidt)
0.5% of polymer (solids content) ad 100% of water
   I) Determination of the Combability

The following instructions describe the procedure for determining the wet and dry combability of hair following treatment with conditioning agents. All measurements were carried out in a climatically controlled room at 65% relative humidity and 21° C.

Instruments Used

Wet combability: Frank tensile/force tester

Dry combability: Diastron force measuring system

Digital balance: (top-pan balance)

Hair:

a) European, bleached: hair tresses from Wernesgrün (bleaching see below)
b) Asiatic, untreated: hair tresses from Wernesgrün with split ends

The following tests were carried out:
wet combability following application of shampoo on European, bleached hair
dry combability following application of shampoo on Asiatic hair
   Pretreatment/Cleansing of the Hair:

Prior to the first use, the Asiatic hair tresses were cleansed in a solvent mixture (ethanol/isopropanol/acetone/water 1:1:1:1) until the hair appeared clean in the dry state (i.e. no longer stuck together). The hair was then washed with sodium lauryl ether sulfate.

The European hair was then treated with a bleaching paste (7.00 g of ammonium carbonate, 8.00 g of calcium carbonate, 0.50 g of Aerosil 200, 9.80 g of hydrogen peroxide (30% strength), 9.80 g of demineralized water). The hair tresses were completely immersed in the bleaching paste to ensure extensive wetting of the entire surface of the hair. The tresses were then wiped between the fingers in order to remove excess bleaching paste. The contact time of the remaining bleach on the hair is matched to the degree of damage required, and is generally 15 to 30 minutes, but can vary depending on the quality of the hair. The bleached hair tresses were then thoroughly rinsed under running tap water (2 minutes) and washed with sodium lauryl ether sulfate. The hair was then dipped briefly into an aqueous acidic solution (e.g. citric acid) because of so-called insidious bleaching, and then rinsed with tap water.

Applications:

The hair tress is dipped for one minute into the surfactant formulation to be tested, shampooed for one minute and then rinsed for one minute under running drinking water (hand-hot).

I) Wet Combability

Determination of the blank value for wet combability: the washed hair was dried overnight in a climatically controlled room. Prior to measurement, it was shampooed twice with Texapon NSO for a total of one minute and rinsed for one minute so that it is definitely wet, i.e. swollen. Prior to the start of the measurement, the tress was precombed until the knots were no longer present in the hair and thus a constant application of force is necessary for the repeated measurement combing. The tress was then fixed to the support and combed using the finely-toothed side of the comb into the finely-toothed side of the test comb. The hair was inserted into the test comb for each measurement uniformly and without tension. The measurement was started and evaluated by means of software (EGRANUDO program, Frank). The individual measurement was repeated 5 to 10 times. The calculated average value was noted.

Determination of the measurement value for wet combability: Following the determination of the blank value, the hair was treated depending on the desired application. The combing force is measured analogously to the blank-value determination.

Evaluation:
Reduction in combing force wet [%]=100−(measurement value*100/blank value)
II) Dry Combability

Determination of the blank value for dry combability: the washed hair was dried overnight in a climatically controlled room. Prior to the start of the measurement, the tress was precombed until knots were no longer present in the hair and thus a constant application of force is necessary for repeated measurement combing. The tress was then fixed to the support and combed into the finely-toothed side of the test comb. The hair was inserted into the test comb for each measurement uniformly and without tension. The measurement was started and evaluated by means of software (mtt-win, DIASTRON). The individual measurement was repeated 5 to 10 times. The calculated average value was noted together with the standard deviation.

Determination of the measurement value for dry combability: following the determination of the blank value, the hair was treated according to the desired application and dried overnight. The combing force was measured analogously to the blank-value determination. Evaluation:
Reduction in combing force dry [%]=100−(measurement value*100/blank value)

The results are summarized in table 2.

TABLE 2
Performance tests with the above mentioned test shampoo

| | | Reduction in | Reduction in | |
|---|---|---|---|---|
| | | combing | combing | Appearance |
| Shampoo | Preparation | force, wet | force, dry | of shampoo |
| example No. | example No. | (Grade/%) | (Grade/%) | solution |
| 6 | 1 | 1-2/53 | 1-2/85 | clear |
| 7 | 2 | 1-2/56 | 1-2/79 | clear |
| 8 | 3 | 1-2/48 | 1-/88 | clear |
| 9 | 4 | 2+/18 | 2/48 | clear |
| 10 | 5 | 2+/29 | 2-3/45 | clear |

Examples 6 to 8 clearly show the excellent properties in the case of the use according to the invention (examples 6 to 8) compared with the known process variant (example 9 to 10). In comparison with this, polymers which are not prepared in the presence of salt do not produce good performance properties. Polymers which are prepared as water-in-water emulsion polymers in the presence of salt exhibit very good performance properties.

A further advantage is that clear formulations are also possible with the polymer according to the invention.

### EXAMPLE 11

### Water-in-Water Emulsion Polymerization of VP, VFA, and QVI in the Presence of Na₂SO₄

Sodium sulfate (77 g), vinylpyrrolidone (80 g), vinylformamide (48 g), quaternized vinylimidazolium (64 g, 50% by weight solution in water) and 0.6 g of triallylamine were dissolved in water (347 g). As protective colloid, 63 g of poly(vinylamine-co-acrylic acid)=1:9) (25% by weight solution in water) were added. The pH was adjusted to a value of 6.75 using 50% sulfuric acid and the emulsion obtained was flushed with nitrogen for about 10 minutes. The free radical initiator (V-50; 0.48 g) was then added and the reaction mixture was heated to 60° C. under a nitrogen atmosphere. After the reaction mixture had been stirred at this temperature for 3 hours, a second portion of V-50 (0.96 g) was added and the mixture was stirred for a further 3 hours at 70° C. and then cooled to room temperature. This gave a white suspension with a polymer content of 23% and a viscosity of 2300 mPas. Dilution of the emulsion with water to 6.5% by weight gave a clear solution of 7500 mPas.

### EXAMPLE 12

### Water-in-Water Emulsion Polymerization of VP, VCap, and QVI in the Presence of Na₂SO₄

Sodium sulfate (77 g), vinylpyrrolidone (88 g), vinylcaprolactam (40 g), quaternized vinylimidazolium (64 g, 50% by weight solution in water) and 0.6 g of triallylamine were dissolved in water (297 g). As protective colloid, 63 g of poly(vinylamine-co-acrylic acid)=1:9) (25% by weight solution in water) were added. The pH was adjusted to a value of 6.75 using 50% sulfuric acid and the emulsion obtained was flushed with nitrogen for about 10 minutes. The free radical initiator (V-50; 0.48 g in 50 ml of water) was then added over 3 hours and the reaction mixture was heated to 65° C. under a nitrogen atmosphere. After the reaction mixture had been stirred at this temperature for 3 hours, a second portion of V-50 (0.96 g) was added, and the mixture was stirred for a further 3 hours at 70° C. and then cooled to room temperature. This gave a white suspension with a polymer content of 23% and a viscosity of 2300 mPas. Dilution of the emulsion with water to 6.5% by weight gave a clear solution of 7500 mPas.

### EXAMPLE 13

### Water-in-Water Emulsion Polymerization of VP, VCap, and QVI in the Presence of NaCl and Na₂SO₄

Sodium sulfate (60 g), sodium chloride (20 g), vinylpyrrolidone (128 g), quaternized vinylimidazolium (64 g, 50% by weight solution in water) and 0.6 g of triallylamine were dissolved in water (347 g). As protective colloid, 63 g of poly(vinylamine-co-acrylic acid)=1:9) (25% by weight solution in water) were added. The pH was adjusted to a value of 6.75 using 50% sulfuric acid, and the emulsion obtained was flushed with nitrogen for about 10 minutes. The free radical initiator (V-50; 0.48 g) was then added and the reaction mixture was heated to 60° C. under a nitrogen atmosphere. After the reaction mixture had been stirred at this temperature for 3 hours, a second portion of V-50 (0.96 g) was added and the mixture stirred for a further 3 hours at 70° C. and then cooled to room temperature. This gave a white suspension with a polymer content of 23% and a viscosity of 2300 mPas. Dilution of the emulsion with water to 6.5% by weight gave a clear solution of 7500 mPas.

Example 14: Clear conditioner shampoo

| % | Ingredient | INCI |
|---|---|---|
| 15.00 | Tego Betaine L 7 | Cocamidopropylbetaine |
| 10.00 | Amphoteric surfactant GB 2009 | Disodium |
| | cocoamphodiacetate | |
| 5.00 | Cremophor PS 20 | Polysorbate 20 |
| 5.00 | Plantacare 2000 | Decyl glucoside |
| 3.00 | Stepan PEG 6000 DS | PEG-150 distearate |
| q.s. | Perfume | |
| q.s. | Preservative | |
| q.s. | Citric acid | |
| 0.1-1.0 | Active substance of conditioner polymer as in Example 1 | |
| 2.00 | Rewopal LA 3 | Laureth-3 |
| ad 100 | Water, demineralized | Aqua dem. |

Example 15: Conditioner shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, |
| | | cocamide MEA, laureth-10 |
| 0.1-1.0 | Active substance of conditioner polymer as in Example 1 | |
| 2.00 | Dimethicone | |
| q.s. | Perfume | |
| q.s. | Preservative | |
| q.s. | Citric acid | |
| 1.00 | Sodium chloride | |
| ad 100 | Water, demineralized | Aqua dem. |

Example 16: Conditioner shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Amidodimethicone | |
| q.s. | Perfume | |
| q.s. | Preservative | |
| q.s. | Citric acid | |
| 1.00 | Sodium chloride | |
| ad 100 | Water, demineralized | Aqua dem. |

Example 17: Conditioner shampoo

| % | Ingredient | INCI |
|---|---|---|
| 40.00 | Texapon NSO | Sodium laureth sulfate |
| 10.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Dow Corning 3052 | |
| q.s. | Perfume | |
| q.s. | Preservative | |
| q.s. | Citric acid | |
| 2.00 | Cocamido DEA | |
| ad 100 | Water, demineralized | Aqua dem. |

Example 18: Conditioner Shampoo

| % | Ingredient | INCI |
|---|---|---|
| 30.00 | Texapon NSO | Sodium laureth sulfate |
| 6.00 | Dehyton G | Sodium cocoamphoacetate |
| 6.00 | Dehyton K | Cocamidopropylbetaine |
| 3.00 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| 2.00 | Dimethicone | |
| q.s. | Perfume | |
| q.s. | Preservative | |
| q.s. | Citric acid | |
| 2.00 | Cocamido DEA | |
| ad 100 | Water, demineralized | Aqua dem. |

Example 19: Anti-dandruff shampoo

| % | Ingredient | INCI |
|---|---|---|
| 40.00 | Texapon NSO | Sodium laureth sulfate |
| 10.00 | Tego Betaine L 7 | Cocamidopropylbetaine |
| 10.00 | Rewopol SB FA 30 | Disodium laureth sulfo- |
| | | succinate |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| 0.50 | Crinipan AD | Climbazole |
| q.s. | Perfume | |
| q.s. | Preservative | |
| q.s. | Citric acid | |
| 0.50 | Sodium chloride | |
| ad 100 | Water, demineralized | Aqua dem. |

Example 20: Shampoo

| % | Ingredient | INCI |
|---|---|---|
| 25.00 | Sodium laureth sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| 2.0 | Cocamido DEA | |
| q.s. | Perfume | |
| q.s. | Preservative | |
| ad 100 | Water, demineralized | Aqua dem. |

Example 21: Shampoo

| % | Ingredient | INCI |
|---|---|---|
| 20.00 | Ammonium laureth sulfate | |
| 15.00 | Ammonium lauryl sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 3 | |
| q.s. | Perfume | |
| q.s. | Preservative | |
| 0.50 | Sodium chloride | |
| ad 100 | Water, demineralized | Aqua dem. |

Example 22: Shampoo

| % | Ingredient | INCI |
|---|---|---|
| 20.00 | Sodium laureth sulfate | |
| 15.00 | Sodium lauryl sulfate | |
| 5.00 | Cocamidopropylbetaine | |
| 2.50 | Euperlan PK 771 | Sodium laureth sulfate, |
| | | glycol distearate, cocamide |
| | | MEA, laureth-10 |
| 0.1-1.0 | Active substance of conditioner polymer as in Example 2 | |
| q.s. | Perfume | |
| q.s. | Preservative | |
| 0.50 | Sodium chloride | |
| ad 100 | Water, demineralized | Aqua dem. |

Example 23: Clear shower gel

| % | Ingredient | INCI |
|---|---|---|
| 40.00 | Texapon NSO | Sodium laureth sulfate |
| 5.00 | Plantacare 2000 | Decyl glucoside |
| 5.00 | Tego Betaine L 7 | Cocamidopropylbetaine |
| 0.1-1.0 | Active substance of conditioner polymer as in Example 2 | |
| 1.00 | D-Panthenol USP | Panthenol |
| q.s. | Perfume | |
| q.s. | Preservative | |
| q.s. | Citric acid | |
| 2.00 | Sodium chloride | |
| ad 100 | Water, demineralized | Aqua dem. |

Example 24: Shampoo

| % | Ingredient | INCI |
|---|---|---|
| 12.00 | Texapon N 70 | Sodium laureth sulfate |
| 1.50 | Plantacare 2000 | Decyl glucoside |
| 2.50 | Dehyton PK 45 | Cocamidopropylbetaine |
| 5.00 | Lamesoft PO 65 | Cocoglucoside glyceryl oleate |
| 2.00 | Euperlan PK 771 | Sodium laureth sulfate, |
| | | glycol distearate, cocamide |
| | | MEA, Laureth-10 |
| 0.1-1.0 | Active substance of conditioner polymer as in Example 1 | |
| q.s. | Preservative | |
| q.s. | Sicovit Sunset | Sunset Yellow C. I. 15 985 |
| | Yellow 85 E 110 | |
| q.s. | Perfume | |
| 1.00 | Sodium chloride | |
| ad 100 | Water, demineralized | |

Example 25: Shampoo

| % | Ingredient | INCI |
|---|---|---|
| 12.00 | Texapon N 70 | Sodium laureth sulfate |
| 1.50 | Plantacare 2000 | Decyl glucoside |
| 2.50 | Dehyton PK 45 | Cocamidopropylbetaine |
| 5.00 | Lamesoft PO 65 | Cocoglucoside glyceryl oleate |
| 2.00 | Euperlan PK 771 | Sodium laureth sulfate, |
| | | glycol distearate, cocamide |
| | | MEA, Laureth-10 |
| 0.1-1.0 | Active substance of conditioner polymer as in Example 1 | |
| q.s. | Preservative | |
| q.s. | Sicovit Sunset | Sunset Yellow C. I. 15 985 |
| | | Yellow 85 E 110 |
| q.s. | Perfume | |
| 1.00 | Sodium chloride | |
| ad 100 | Water, demineralized | |

We claim:

1. The use of crosslinked cationic polymers preparable by polymerization of
a) 1 to 99.9% by weight, based on the total amount of monomers used for the preparation of the polymer, of at least one cationic or cationogenic vinyl group-containing monomer chosen from the group consisting of N-vinylimidazoles, diallylamines, dialkylaminoalkyl(meth)acrylamides and dialkylaminoalkyl(meth)acrylamides and dialkylaminoalkyl (meth)acrylates,
b) 0 to 99% by weight, based on the total amount of monomers used for the preparation of the polymer, of at least one neutral or basic water-soluble monomer different from (a),
c) 0 to 50% by weight, based on the total amount of monomers used for the preparation of the polymer, of at least one unsaturated acid or one unsaturated anhydride,
d) 0 to 50% by weight of at least one free-radically copolymerizable monomer different from (a), (b) or (c); and
e) 0.05 to 10% by weight, based on the total amount of monomers used for the preparation of the polymer, of at least one crosslinking monomer with at least two ethylenically unsaturated, nonconjugated double bonds,
where the amounts a) to e) are chosen such that the resulting polymer, optionally after quaternization or protonation, has an overall positive charge,
in water in the presence of
f) 1 to 100% by weight of the saturation amount in the reaction medium of one or more organic or inorganic salts, and
g) 0.1 to 30% by weight, based on the total weight of the dispersion, of at least one water-soluble protective colloid with a composition different from a) to e), and
subsequent at least partial quaternization for cases where the monomer (a) is not quaternized,
in cosmetics.

2. The use as claimed in claim 1 in hair cosmetics.

3. The use as claimed in claim 1, wherein the free-radically polymerizable vinyl group-containing cationic monomer used is at least one N-vinylimidazole derivative of the formula (I), 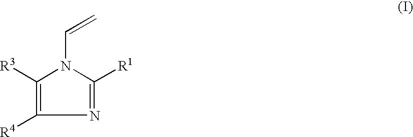
in which the radicals R¹ to R³, independently of one another, are hydrogen, C₁-C₄-alkyl or phenyl.

4. The use as claimed in claim 1, wherein the free-radically polymerizable vinyl group-containing cationic monomer used is at least one diallylamine derivative of the formula (II), 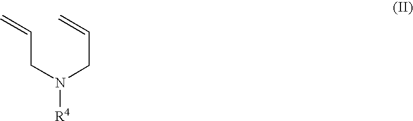
in which the radical R⁴ is C₁-C₂₄-alkyl.

5. The use as claimed in claim 1, wherein the free-radically polymerizable vinyl group-containing cationic monomer used is at least one dialkylaminoalkyl(meth)acrylamide and dialkylaminoalkyl (meth)acrylate of the formula (III), 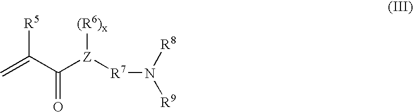
in which R⁵ and R⁶, independently of one another, are hydrogen or methyl, Z is a nitrogen atom where x=1 or an oxygen atom where x=0, R⁷ is a linear or branched C₁-C₂₄-alkylene radical, and R⁸ and R⁹, independently of one another, are a C₁-C₂₄-alkylene radical.

6. The use as claimed in claim 1, where the monomer (b) used is at least one N-vinyllactam.

7. The use as claimed in claim 1 as conditioning agent or thickener.
